(19) United States
(12) Patent Application Publication
(10) Pub. No.: US 2003/0092040 A1
(43) Pub. Date: May 15, 2003

(54) System and method for identifying a genetic risk factor for a disease or pathology

(76) Inventors: Joel S. Bader, Stamford, CT (US); Pak Sham, London (GB)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/215,280

(22) Filed: Aug. 8, 2002

Related U.S. Application Data
(60) Provisional application No. 60/310,796, filed on Aug. 8, 2001.

(51) Int. Cl. C12Q 1/68; G06F 19/00; G01N 33/48; G01N 33/50

(52) U.S. Cl. 435/6; 702/20

(57) ABSTRACT

The invention relates to systems and methods for detecting nucleic acid molecule encoding GENE-X having nucleotide polymorphisms indicative of increased risk for DISEASE-X. The invention also relates to a method for identifying individuals who are carriers of the genetic risk factor or are at increased risk. The method includes obtaining a biological sample from an individual and testing the individual for the nucleotide polymorphism, wherein the disease risk may increase with the expression of ALLELE-X.

### RELATED APPLICATION

This application claims priority from U.S. Provisional Patent Application Serial No. 60/310,796 filed Aug. 8, 2001, the entirety of which is hereby incorporated by reference.

### FIELD OF THE INVENTION

The present invention relates to systems and methods that utilize statistical means for analyzing biological samples for genetic polymorphisms and other genetic markers for disease states or disorders.

### BACKGROUND OF THE INVENTION

Gene expression varies within populations and even within what appears to be a homogeneous population. The most challenging aspects of presenting gene expression data involve the quantification and qualification of expression values, including standard statistical significance tests and confidence intervals. The current state of the art in array-based studies precludes obtaining standard statistical indices (e.g., confidence intervals, outlier delineation) and performing standard statistical tests (e.g., t-tests, analyses-of-variance) that are used routinely in other scientific domains, because the number of replicates typically present in such studies would ordinarily be considered insufficient for these purposes. Thus, statistical indices and tests are required so estimates can be made about the reliability of observed differences between expression conditions. The key question in these kinds of comparisons is whether it is likely that observed differences in measured values reflect random error only or random error combined with treatment effect (i.e., “true difference”), and whether these polymorphisms have any relevance as markers for disease states or pathological conditions.

### SUMMARY OF THE INVENTION

The present invention is directed to a system and methods for determining the risk of an individual or relative of the individual, of developing a disease or pathological disorder, wherein the disease or disorder is correlated with a genetic locus, and the disease phenotype is correlated with gene polymorphisms at that locus.

In one aspect, the invention includes a system for detecting a genetic risk factor for a disease or pathological condition. The system includes hardware and software modules for data management, e.g., a data input means, a data storage means, a data retrieval means, and a data output means, as well as an instruction set and processing means. In one embodiment, the instruction set includes an input module. The input module instructs the system in entering data in computer readable format. The data includes patient sample information and reference sample information. In one embodiment, the sample information includes patient medical histories, genotype and phenotype information for disease markers, population information for allele frequencies, ethnicity, and general medical information. In one embodiment, a selection module is incorporated into the system, thus instructing the system to select and read entered data, user defined or obtained from databases. In another embodiment, the invention includes an analyzing module. The analyzing module instructs the system to perform biostatistical analyses of the entered data, for example, the patient sample information and reference sample information, and thereby detects statistically significant similarities or differences between the patient sample information and the reference sample information. In yet another embodiment, the system includes an association detection module. The association detection module instructs the system to correlate statistically significant similarities or differences between the patient sample information and the reference sample information with data relating to a pathological phenotype. In still another embodiment, the system includes a presenting module. The presenting module instructs the system to present to the user, the statistically significant similarities or differences between the patient sample information and the reference sample information, and the data relating to a pathological phenotype. The user uses the present system to detect and assess the patient's genetic risk factor for the disease.

In another aspect, the invention relates to a processor readable medium having program code for executing specific functions. In one embodiment, the program code causes a processor to select and read entered patient derived data, including but not limited to, patient sample information and reference sample information. In another embodiment, the program code causes the processor to perform biostatistical analyses of the entered data, thereby detecting statistically significant similarities or differences between the patient sample information and the reference sample information. In yet another embodiment, the program code causes the processor to correlate statistically significant similarities or differences between the patient sample information and the reference sample information with data relating to a pathological phenotype. In still another embodiment, the program code causes the processor to present to the user, the statistically significant similarities or differences between the patient sample information and the reference sample information, and the data relating to a pathological phenotype, thus permitting the user to detect the patient's genetic risk factor for the disease.

In still another aspect, the invention provides a method for detecting a genetic risk factor for a disease. In this aspect, patient derived biological sample are obtained, wherein the patient derived sample contains a detectable marker correlated with a disease state or pathological condition. Such disease markers are well known in the art. In one embodiment, data is obtained from the biological sample, such as but not limited to patient sample information, for example, a polymorphism in the nucleotide sequence of a gene marker, the determination of a polymorphism being made by comparison of the patient derived sample relative to the sequence of a wild-type marker, i.e., a sample sequence obtained from a healthy individual. In this embodiment, the polymorphism is correlated with a disease state or pathological condition, and detecting an association between the patient sample information and a disease state, is thus predictive of a genetic risk factor for the patient to develop the disease. In still another embodiment, detecting the association between the patient sample and a disease state is accomplished by performing Hardy-Weinberg tests, association tests (such as quantitative trait locus analysis (QTL)), Chi-square analysis, and other biostatistical manipulations on the patient sample information.

In one aspect of the invention, patient sample information at a gene locus is obtained by genotyping methods such as but not limited to oligonucleotide ligation, direct sequencing, mass spectroscopy, real time kinetic PCR, hybridization, pyrosequencing, fragment polymorphisms, and fluorescence depolarization. This patient sample information is communicated to the system of the present invention, in the form of processor readable program code, which allows a user to input patient sample information obtained by these genotyping methods In another aspect, patient derived biological samples are obtained from tissues and fluids containing any nucleated cell, such as but not limited to blood, hair folicles, buccal scrapings, saliva, organ biopsies, and semen. This patient sample information is communicated to the system of the present invention, in the form of processor readable program code, which allows a user to input patient sample information obtained by these techniques. Reference sample information is input into the system using similar means.

In another aspect of the invention, the patient sample information is predictive of a risk for the patient to develop a genetic disease. The patient sample information is communicated to the system in the form of processor readable program code for causing a processor to perform biostatistical analyses, which detects a polymorphism in a patient gene sequence that is predictive of a risk for the patient to develop a genetic disease. In still another aspect, the system provides a method where patient sample information is predictive of a risk for one or more offspring of the patient to develop a genetic disease. In yet another aspect of the invention, the system provides a method where patient sample information is predictive of a risk for siblings of the patient to develop a genetic disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

Definitions

The term “AA1-LOC-AA2” as used herein refers to a patient derived variant polypeptide sequence, where “AA1” and “AA2” are first and second amino acids flanking a third amino acid “LOC”. AA1 and AA2 have identity to, or are conservative substitutions of, first and second amino acids contained in a three amino acid fragment of a reference wild-type polypeptide, having the sequence (AA1-X-AA2), where X is an amino acid present in the wild-type polypeptide sequence, and where a change in X to LOC is indicative of or correlates with a genetic risk factor for a disease or a pathology.

The term “ALIGNMENT” as used herein refers to a sequence alignment between a reference sequence and a patient derived variant polypeptide sequences, or a DNA sequence encoding the same.

The term “ALLELE” as used herein refers to the polynucleotide sequence of a gene locus. The tern “HAPLOTYPE” as used herein refers to the presence of a particular variant allele, for which the polynucleotide sequence is a marker for a disease risk or pathological condition, and which encodes a variant polypeptide with altered function relative to the wild-type, where the altered function is indicative of or correlates with a genetic risk factor for a disease or a pathology. The term “SNP” as used herein refers to single nucleotide polymorphisms and/or multiple nucleotide variations within an allele, resulting in a change in haplotype at that allele. The term “LDGROUP ” as used herein refers to other nucleotide variants that are in linkage disequilibrium with the SNP and may also be used as markers for disease or pathological conditions. The term “REFDNASEQ” as used herein refers to a reference DNA sequence, obtained from healthy tissues and used a negative control, or from diseased or pathological tissues, and used as a positive control for a genetic risk factor for a disease or a pathology.

The term “DISEASE” as used herein refers to a condition characterized by a pathological phenotype, where the pathological phenotype is related to the overexpression or underexpression of a gene product having one or more allelic polymorphisms, i.e., haplotypes indicative of the pathological phenotype. Pathologies, diseases, disorders and condition and the like include, but are not limited to e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases, and various cancers, as well as conditions such as transplantation and fertility.

The term “ETHNICITY” as used herein refers to the ethnic background of a patient, relevant in that such an individual with such ethnic background demonstrates a higher probability relative to a population with a different ethnic background, for one or more genetic polymorphisms within a gene locus that are correlated with disease risk. Ethnicity is important in evaluating a patient's genetic predispositions to certain diseases as it often suggestive of a particular genetic predisposition of a subpopulation to a disease phenotype, such as Tay-Sachs disease, which is more common to persons of Eastern European ancestry, or Sickle Cell Anemia, which is more common to persons of African ancestry.

The term in “SEQID” as used herein refers to a sequence identifier.

The present invention relates to systems and methods for correlating the presence of allelic polymorphisms, or haplotypes, with disease states, thereby providing methods for evaluating the risk of an individual patient for developing a particular pathological condition, or to monitor the course of a disease state in an individual. The invention relates to the detection of a human gene obtained from a patient sample, generically referred to herein as GENE-X as well as systems and methods for identifying nucleic acid and amino acid sequences having polymorphisms of GENE-X, where the presence or absence of polymorphisms are useful for identifying individuals who are affected by, predisposed to, at risk for, or are carriers of DISEASE-X.

Allelic polymorphisms are frequently seen in population genetics studies, for example, where the patient has a particular ethnicity, generically referred to as ETHNICITY-X, the individuals of which may have a propensity relative to other ethnic backgrounds for polymorphisms of GENE-X, e.g., sickle cell anemia, Tay-Sachs disease, or other heritable disorders (see, D. S. Falconer and T. F. C. Mackay, Introduction to quantitative genetics, 4^{th} edition, Prentice Hall, New York, 1996, incorporated by reference). A non-limiting example of several genes and their associated disease states is given at Example A as Table A.

A polymorphism in the gene encoding a particular GENE-X in humans is detected. Background information is obtained from a patient, for example, ethnicity, identified as ETHNICITY-X. This information is analyzed using the system and methods of the present invention, and individuals who are afflicted by, predisposed to, or carriers of DISEASE-X are identified, by detecting the presence or absence of the polymorphism using simple nucleic acid based diagnostic tests. Therefore, individuals are identified for more frequent monitoring for the development of a pathological condition, and earlier or more aggressive intervention in the treatment of a disease state.

Identification of Single Nucleotide Polymorphisms in Nucleic Acid Sequences

A variant sequence is an allelic polymorphism, and can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a-substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling™ assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (see, Alderborn et al., Determination of Single Nucleotide Polymorphisms by Real-time Pyrophosphate DNA Sequencing. Genome Research. 10 (8) 1249-1265, 2000, incorporated herein by reference). Other well-known methods of determining a polynucleotide sequence are appropriate for detecting allelic polymorphisms between patient derived and reference samples, and include sequencing by hybridization, dideoxy sequencing, the Sanger method, spectroscopic and other methods. These are considered to be within the scope of the invention.

Determining Homology Between Two or More Sequences

A variant haplotype is determined by comparing a patient derived sample sequence against one or more reference samples, and evaluating the nucleic acid homology of the patient and reference samples for polymorphisms. Reference samples comprise samples of biological materials that are positive or negative for a polypeptide or polynucleotide encoding same, that is associated with the disease state or pathological condition. In one embodiment, a reference sample is obtained from healthy cells or tissues, where no disease state or pathological phenotype is observed, and where the tissues exhibit normal levels of gene expression of the wild-type polypeptide. In another embodiment, a reference sample is obtained from pathological cells or tissues, where one or more disease states or pathological phenotypes are observed, and where the tissues exhibit aberrant levels of gene expression of the variant polypeptide relative to healthy tissues. A non-limiting example of this includes staged cancer tissues. Thus reference samples provide qualitative comparisons with patient derived samples. To determine the percent homology of amino acid sequences or of nucleic acid sequences, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between the aligned sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence

The term “sequence identity” as used herein refers to the degree to which two polynucleotide or polypeptide sequences are identical on a residue-by-residue basis over a particular region of comparison. The term “percentage of sequence identity” as used herein is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once an allele (or a portion of the sequence) of a gene implicated in a disease state has been isolated, this sequence can be used to map the location of the allele on a chromosome. This process is called chromosome mapping. The mapping of the sequences to chromosomes is an important first step in correlating these sequences with genes associated with disease.

Briefly, genes can be mapped to chromosomes by preparing PCR primers (preferably 15-25 bp in length) from known polypeptide or polynucleotide sequences. Computer analysis of the target sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the target sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the target sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colchicine that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDLIAN INHERITANCE IN MAN, (available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, polymorphic differences in the DNA sequences between individuals affected and unaffected with a disease associated with the haplotype, can be determined. If a polymorphism is observed in some or all of the affected individuals but not in any unaffected individuals, then the polymorphism is likely to be a causative agent of the particular disease, or a marker for a pathological condition associated with the disease.

Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a polymorphism and to distinguish polymorphisms from other variations such as mutations.

Panels of corresponding nucleic acid sequences from individuals, prepared in this manner, can provide unique individual identifications, as each individual will have a unique set of such sequences due to allelic differences. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

A polymorphism is used for prediction of a disease state as polymorphisms acan be markers for disease states, such that the presence of a polymorphism increases the probability the subject will acquire the disease. Alternatively, a polymorphism can indicate a decrease in the probability the subject will acquire the disease. Polymorphisms can also indicate familial predispositions to or resistance to disease states, for example, that a sibling or offspring of a patient will have a probability for developing the disease.

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to assess an individuals risk for a pathological condition, or to monitor treatment of an individual undergoing therapy for the disease.

Accordingly, one aspect of the invention relates to diagnostic assays for determining polypeptide and/or nucleic acid expression or activity, in the context of a biological sample (e.g., blood, serum, cells, tissue) to thereby determine whether an individual carrying GENE-X is afflicted with a disease or disorder, or is at risk of developing a disorder associated with aberrant expression or activity of a particular haplotype of GENE-X. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipideimias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with a particular GENE-X haplotype resulting from aberrant polypeptide or polynucleotide expression or activity. For example, mutations in a gene locus can be assayed in a patient derived biological sample. Such assays can be compared against reference samples, and used for prognostic or predictive purpose to thereby prophylactically treat an individual prior to the onset of a disorder characterized by or associated with the variant polypeptide or nucleic acid having aberrant biological activity.

Another aspect of the invention provides methods for determining GENE-X polypeptide or nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of an allele in clinical trials.

Diagnostic Assays

An exemplary method for detecting the presence or absence of GENE-X in a biological sample involves obtaining a biological sample from a test subject and contacting the biological sample with a compound or an agent capable of detecting, GENE-X protein or nucleic acid (e.g., mRNA, genomic DNA) that encodes GENE-X protein such that the presence of GENE-X is detected in the biological sample. An agent for detecting GENE-X mRNA or genomic DNA is a labeled nucleic acid probe capable of hybridizing to GENE-X mRNA or genomic DNA. The nucleic acid probe can be, for example, a full-length GENE-X nucleic acid, or a portion thereof, such as an oligonucleotide of at least 15, 30, 50, 100, 250 or 500 nucleotides in length and sufficient to specifically hybridize under stringent conditions to GENE-X in RNA or genomic DNA. Other suitable probes for use in the diagnostic assays of the invention are described herein.

An agent for detecting GENE-X protein is an antibody capable of binding to GENE-X protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Any nucleated cell can be used, for example but not limited to blood, hair follicles, buccal scrapings, saliva, semen, and organ biopsies. That is, the detection method of the invention can be used to detect GENE-X mRNA, protein, or genomic DNA in a biological sample in vitro as well as in vivo For example, in vitro techniques for detection of GENE-X mRNA include Northern hybridizations and in situ hybridizations. In vitro techniques for detection of GENE-X protein include enzyme linked immunosorbent assays (ELISAs), Western blots, immunoprecipitations, and immunofluorescence. In vitro techniques for detection of GENE-X genomic DNA include Southern hybridizations. Furthermore, in vivo techniques for detection of GENE-X protein include introducing into a subject a labeled anti-GENE-X antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting GENE-X protein, mRNA, or genomic DNA, such that the presence of GENE-X protein, in RNA or genomic DNA is detected in the biological sample, and comparing the presence of GENE-X protein, mRNA or genomic DNA in the control sample with the presence of GENE-X protein, mRNA or genomic DNA in the test sample.

The invention also encompasses kits for detecting the presence of GENE-X in a biological sample. For example, the kit can comprise: a labeled compound or agent capable of detecting GENE-X protein or mRNA in a biological sample; means for determining the amount of GENE-X in the sample; and means for comparing the amount of GENE-X in the sample with a standard. The compound or agent can be packaged in a suitable container. The kit can further comprise instructions for using the kit to detect GENE-X protein or nucleic acid.

Prognostic Assays

The diagnostic methods described herein can furthermore be utilized to identify subjects having or at risk of developing a disease or disorder associated with aberrant GENE-X expression or activity. For example, the assays described herein, such as the is preceding diagnostic assays or the following assays, can be utilized to identify a subject having or at risk of developing a disorder associated with GENE-X protein, nucleic acid expression or activity. Alternatively, the prognostic assays can be utilized to identify a subject having or at risk for developing a disease or disorder. Thus, the invention provides a method for identifying a disease or disorder associated with aberrant GENE-X expression or activity in which a test sample is obtained from a subject and GENE-X protein or nucleic acid (e.g., mRNA, genomic DNA) is detected, wherein the presence of GENE-X protein or nucleic acid is diagnostic for a subject having or at risk of developing a disease or disorder associated with aberrant GENE-X expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant GENE-X expression or activity. For example, such methods can be used to determine whether a subject can be effectively treated with an agent for a disorder. Thus, the invention provides methods for determining whether a subject can be effectively treated with an agent for a disorder associated with aberrant GENE-X expression or activity in which a test sample is obtained and GENE-X protein or nucleic acid is detected (e.g., wherein the presence of GENE-X protein or nucleic acid is diagnostic for a subject that can be administered the agent to treat a disorder associated with aberrant GENE-X expression or activity).

The methods of the invention can also be used to detect genetic lesions in a GENE-X gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a GENE-X-protein, or the misexpression of the GENE-X gene. For example, such genetic lesions can be detected by ascertaining the existence of at least one of: (i) a deletion of one or more nucleotides from a GENE-X gene; (ii) an addition of one or more nucleotides to a GENE-X gene; (iii) a substitution of one or more nucleotides of a GENE-X gene, (iv) a chromosomal rearrangement of a GENE-X gene; (v) an alteration in the level of a messenger RNA transcript of a GENE-X gene, (vi) aberrant modification of a GENE-X gene, such as of the methylation pattern of the genomic DNA, (vii) the presence of a non-wild-type splicing pattern of a messenger RNA transcript of a GENE-X gene, (viii) a non-wild-type level of a GENE-X protein, (ix) allelic loss of a GENE-X gene, and (x) inappropriate post-translational modification of a GENE-X protein. As described herein, there are a large number of assay techniques known in the art which can be used for detecting lesions in a GENE-X gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e g., Landegran, et al., 1988. Science 241: 1077-1080; and Nakazawa, et al., 1994. Proc. Natl. Acad. Sci. USA 91: 360-364), the latter of which can be particularly useful for detecting point mutations in the GENE-X-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a GENE-X gene under conditions such that hybridization and amplification of the GENE-X gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a GENE-X gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in GENE-X can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in GENE-X can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the GENE-X gene and detect mutations by comparing the sequence of the sample GENE-X with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the GENE-X gene include methods in which protection from cleavage agents is used to detect mismatched bases in RNA/RNA or RNA/DNA heteroduplexes. See, e.g., Myers, et a/., 1985. Science 230: 1242. In general, the art technique of “mismatch cleavage” starts by providing heteroduplexes of formed by hybridizing (labeled) RNA or DNA containing the wild-type GENE-X sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GENE-X cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a GENE-X sequence, e.g., a wild-type GENE-X sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in GENE-X genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control GENE-X nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313:495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In Such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a GENE-X gene.

Furthermore, any cell type or tissue, preferably peripheral blood leukocytes, in which GENE-X is expressed may be utilized in the prognostic assays described herein. However, any biological sample containing nucleated cells may be used, including, for example, buccal mucosal cells.

Biostatistical Analysis

Data Collection

The invention corresponds to a system and method for detection or identification of allotypic variations among individuals, where the presence of a GENE-X polypeptide or polynucleotide encoding the same, is detected as described above, and a determination is made as to whether the individual is polymorphic for GENE-X relative to one or more reference samples or in view of known medical information about GENE-X and GENE-X polymorphisms. The entire GENE-X need not be identified, rather detection of fragments indicative of pathological conditions is sufficient, for example where nucleic acid polymorphisms are indicative of a predisposition or resistance to a disease state, the GENE-X sequence and probes or primers designed to amplify it or hybridize to it must be long enough to serve in genotyping assays that provide an indication of the sequence of GENE-X, and to reveal polymorphisms in the open reading frame or in regulatory sequences. Thus, the nucleic acid molecules need not be identical to the entire coding and non-coding sequence of GENE-X, excluding the polymorphism. Instead, the molecules need to have sufficient identity to fragments of GENE -X such that the nucleic acid molecule may be used to differentiate between the presence or absence of a nucleic acid polymorphism.

The invention also relates to a system and method for identifying individuals, particularly of ETHNICITY-X, who are affected by, predisposed to, or carriers of DISEASE-X caused by presence of the ALLELE-X variant in their genome. The method includes obtaining a biological sample from an individual and testing the sample for ALLELE-X, wherein the allele dose correlates with increased disease risk.

Data from one or more subject patients are obtained, including a medical history for the patient under study and more preferably including the patient's family medical histories and ethnicity information. A gene locus implicated in a disease or disorder is selected for further study. Patient derived samples are compared with reference samples, or existing medical information and an association with a disease state or risk factor for developing a disease state can be determined by methods known to medical professionals or others similarly skilled in biological, biostatistical or medical arts. The American Type Culture Collection provides tissue samples that can be used as reference samples, as well as information on the tissue sources and pathological phenotypes. Other sources for medical information include the PUBMED database, from the National Archives of Medicine. Other such databases for specific diseases also exist, such as for specific cancers, and are known to skilled artisans.

Population, Clinical Measurements, and Genotypes

Hardy-Weinberg Tests

Hardy-Weinberg equilibrium (HWE) relates genotype frequencies to allele frequencies under general assumptions of an equilibrium population. Violations of HWE may indicate selection against the minor allele and population stratification. Selection against the minor allele occurs when the minor allele detracts from evolutionary fitness and may result in having fewer homozygotes than would be expected by chance. Population stratification arises when the population being studies is actually a mix of sub-populations with different frequencies of allele A. Stratification results in having more homozygotes than would be expected by chance. Stratification may increase the false-positive and false-negative rates for between-family tests but does not affect within-family tests (see below). Thus, if stratification is indicated, it is preferable to perform only within-family tests.

The HW1 test is the standard test, but it is not accurate when the smallest category, typically N(AA), has fewer than 5 individuals. The HW2 test is more robust but can be less sensitive for rare alleles. If there is significant deviation from HWE, the sign of [N(AA)+N(BB)]−[n(AA)+n(BB)] indicates the reason: positive values indicate stratification and negative values indicate selection against the minor allele.

To perform Hardy-Weinberg analysis, an individual is selected at random from each monozygous (MZ) and dizygous (DZ) pair to yield a total of N=nUnrel+nMZ+nDZ unrelated individuals. The counts of individuals with AA, AB, and BB genotypes in this population were termed N(AA), N(AB), and N(BB), respectively, and the allele frequency p was calculated as:

p=[N(AA)+0.5 N(BB)]/N

Next, the counts of individuals expected for each genotype under the null hypothesis of HWE is calculated as:

n(AA)=p²N

n(AB)=2pqN

n(BB)=q²N

Finally, two test statistics are calculated:

HW1=[N(AA)−n(AA)]²/n(AA)+[N(AB)−n(AB)]²/n(AB)+[N(BB)−n(BB)]²/n(BB)

HW2={[N(AA)+N(BB)]−[n(AA)+n(BB)]}²/{n(AA)+n(BB)}+[N(AB)−n(AB)]²/n(AB)

Under the null hypothesis, both HW1 and HW2 follow χ² distributions with 1 degree of freedom. The critical values of χ² for p-values of 0.05 and 0.01 are 3.84 and 6.63 respectively. Values of χ² larger than these indicate a 5% chance or a 1% chance of the HW assumptions being satisfied.

Association Tests

Association tests were based on a genetic model for the marker as a quantitative trait locus (QTL):

X_{fi}=Y_{f}+Y_{fi}+m(G_{fi})

where X_{fi} is the phenotypic value of individual i in family f, Y_{f} represents the contribution to X_{fi} from shared genetic and environmental effects excluding effects from the QTL, Y_{fi} represents the non-shared contributions excluding the QTL, and m(G_{fi}) represents the mean effect from the QTL and depends only on the genotype G_{fi}, with:

m(AA)=a−c

m(AB)=d−c

m(BB)=−a−c

where the constant c is defined as:

(p−q)a+2pqd.

Instead of testing for the significance of both a and d, the additive contribution from the allele to the phenotype was emphasized by testing the significance of the regression coefficient b in the model

X₁=Y₁+a+b p₁

where X₁ is the phenotypic value for sample i, Y₁ represents the contributions to the phenotype excluding the QTL for sample i, and p₁ is the allele frequency for sample i.

Since p₁ takes a discrete number of values, the tests were performed by calculating the mean and standard error of X₁ for each value of p₁, then performing a regression test of the binned values to obtain b and its sampling standard deviation s. Under the null hypothesis of no association, b/s follows a standard normal distribution. The p-value for a significant association was calculated from a two-sided test of b/s.

For the mean, difference, and total tests, the tern b is related to the parameters of the genetic model as:

b=2[a−(p−q)d]

The effect size was reported as the quantity a assuming additive inheritance:

(d=0)

then taking the ratio of a to the standard deviation of the trait value.

A multiple testing correction was applied by requiring a p-value of less than approximately 10⁻³ for a significant test.

For further information on biostatistical analysis for particular marker/trait combinations, see, G. W. Snedecor and W. G. Cochran, Statistical Methods, 8^{th} edition, Iowa State University Press, Ames, Iowa, 1989, incorporated herein by reference. These types of analyses are appropriate for use in developing such algorithms for use on systems described, and are considered to be within the scope of the invention.

Computer System for Performing Statistical Analysis of Genetic Risk Factors

In one aspect, a system is provided for performing biostatistical analysis, i.e., calculations on information obtained from patient samples, and correlating the patient information with medical information, such that the patient's risk for developing a disease state or pathological condition can be determined or otherwise predicted. The system comprises modules for data management, e.g., a data input means, a data storage means, a data retrieval means, and a data output means, as well as an instruction set and processing means. Processors appropriate for the system include any processors capable of recognizing an instruction set written in an appropriate language, for example but not limited to PowerPC based Apple® computers, Pentium® or similar PC type computers, SUN® or Silicon Graphics® workstations, or systems running LINUX or UNIX. The system is computer based, and may involve a standalone computer or one or more networked computers, for example packet-switched networks running relational database programs. In a currently preferred embodiment, the system is a plurality of computers in communication with a network, and analysis can be performed anywhere on the network.

The instruction set comprises a computer readable algorithm comprising the aforementioned statistical equations, which is stored in computer readable media as part of a program written in a suitable language, for example C, C++, UNIX, FORTRAN, BASIC, PASCAL, or the like. The program provides the processor with instructions for performing biostatistical analysis on the input data, as well as other functional elements contained in one or more modules or subroutines (e.g., relational database capabilities, search features, and other user defined functions). An example of such an algorithm is provided as Example B. The algorithm includes input modules for entering data into the system in computer readable format; a selection module instructing the system to select and read data entered relating to one or more patients or biological samples, or from plurality of data sources input by the user or by automated means; an analyzing module instructing the system to perform biostatistical analyses of the entered data further comprising the patient sample information and reference sample information, thereby detecting statistically significant similarities or differences between the patient sample information and the reference sample information; an association detection module instructing the system to correlate statistically significant similarities or differences between the patient sample information and the reference sample information with data relating to a pathological phenotype. An association detection may be employed as a subroutine in the instruction set, which module detects an association between at least one genetic locus and at least one phenotype by measuring the allele frequency difference between the samples. This detection is performed by one or more user selectable programmable formula(s). In certain embodiments, association detection would be performed automatically without user intervention, and would be based on pre-determined routines; and a presenting module instructing the system to present to the user, the statistically significant similarities or differences between the patient sample information and the reference sample information, and the data relating to a pathological phenotype, wherein the user detects the patient's genetic risk factor for the disease.

In one aspect, the system includes an input module. Users of the system enter data into the system in computer readable format, which can be stored in RAM or ROM, or a more permanent storage medium such as a disk or tape drive. The information entered through the input module is thus accessible to the system processor. Examples of data entered into the system through an input module are data comprising patient sample information and reference sample information, which include, but are not limited to patient medical history information, genetic information, information about the patient's family and their medical histories, polynucleotide sequence information for one or more gene loci or regulatory elements, genetic disease markers, and medical data from public databases, such as PLUMBED, BLAST, SWISSPROT and similar public and private databases. Users enter information through common data entry means such as a keyboard, GUI, mouse, voice commands, wireless devices and remote data links.

In one aspect, the system includes a selection module. The selection module instructs the system to select and read entered data. Information input by a user is retrieved from memory and communicated to the processor through a processor readable routine or program. These processor readable routines or programs would communicate with one or more user interfaces, preferably a graphical user interface. A user would be able to enter data in one or more interfaces, such as information obtained from a patient sample, or information obtained from the cells and tissues of healthy or disease afflicted individuals for use as reference samples. The user selected data communicated to the system by the selection module is stored by the system in memory for processing.

The system further includes an analyzing module. The analyzing module is an instruction set instructing the system to perform biostatistical analyses of the entered data Differences and similarities between the patient sample information and reference sample information are calculated according to the biostatistical algorithms disclosed herein., i.e., association tests, Hardy-Weinberg tests, chi square tests, and other statistically relevant bioinformatic calculations, thereby detecting statistically significant similarities or differences between the patient sample information and the reference sample information.

The invention further includes an association detection module. The association detection module instructs the system to correlate statistically significant similarities or differences between the patient sample information and the reference sample information with data relating to a pathological phenotype. The association detection module further instructs the processor to execute a program for selecting information about phenotypic or genotypic similarities, and known medical information about these phenotypes or genotypes from public and private databases. For example, the phenotypic database could comprise at least one unique individual identification number and one or more phenotypic values for each individual. In a specific embodiment, a phenotypic database would include other modifiable user input information that is related to a phenotype of one or more individuals. In certain embodiments, selection of individuals would be performed automatically without user intervention, based on pre-determined routines. In a parallel embodiment, phenotypic data that is input into the selection module analysis is derived from a pre-existing database. Computer readable program code would be used to select individuals with at least one pre-determined value.

The system further includes a presenting module. The presenting module instructs the system to present to the user, the statistically significant similarities or differences between the patient sample information and the reference sample information, and the data relating to a pathological phenotype, wherein the user detects the patient's genetic risk factor for the disease. The output of the computer system can be represented in a word processing text file, formatted in commercially-available software such as WordPerfect® and Microsoft Word®, or represented in the form of an ASCII file, stored in a database application, such as DB2, Sybase, Oracle, or the like. A skilled artisan can readily adapt any number of data processor structuring formats (e.g. text file or database) in order to obtain computer readable medium having recorded thereon the expression information of the present invention. The system having provided to the user information pertinent to any statistically relevant correlations or associations between a trait developed from a medical history or genetic analysis, as well as known information about disease phenotypes, thus permits a medical professional or other skilled artisan to assess a risk factor for the patient, whereby the patient's propensity to develop the pathological condition is determined.

For example, a patient provides a tissue sample which is used to screen for the presence of disease using the gene marker GENE-X. By way of illustration, hybridization experiments are performed by any method known to one skilled in the art, and the information obtained from the results of a hybridization is used to determine polymorphisms between a patient derived and reference sample for GENE-X. In this illustration wild-type GENE-X is not implicated in disease, but a polymorphism of GENE-X exists which provides a disease phenotype, that can be exacerbated in individuals with a certain ethnicity, as in the case where loss of gene function can be partially compensated by other genes. The polymorphism has the polypeptide sequence AA1-LOC-AA2 at a region of the sequence, where AA1 and AA2 are identical to their corresponding wild-type amino acid sequences and “LOC” is an amino acid substitution resulting from a single nucleotide polymorphism SNP-X, in the gene encoding the polymorphic GENE-X.

Data is obtained from the patient, including data about the patient's family medical histories, occurrences of disease states in related family members, or prior episodes of the disease state in the patient, as well as gene sequence information from GENE-X, which is a disease state marker. Such data is entered as described by a user into the present system, for example, into a personal computer capable of reading and processing instruction sets written in a computer readable language such as C, (see, Example B) where the data is stored in memory and manipulated by the processor using the algorithm or instruction set comprising the modules described above. The system analyzes and detects potential disease associations based on the patient information compared to reference information. This information may be stored in one or more databases. A typical database may also contain genomic or proteomic information, patient histories, and annotations for each disease marker. In a currently preferred embodiment, a relational database is used to store and cross-reference entered data, for example such as the SPOTFIRE™ relational database. Genotypic and phenotypic databases of the present invention are proprietary or are open source (e.g., GenBank, EMBL, SwissProt), or any combination of proprietary and open source databases. Furthermore, genotypic and phenotypic databases of the present invention are true object oriented, true relational or hybrid of object and relational databases. Which genotypic or phenotypic database to use, or whether to generate a genotypic or phenotypic database de novo, would be well known to one skilled in the art.

The system includes a means for providing output information, thus making it available to the user, which is visualized by an output device such as a graphical user interface, or a printed copy. The output information permits a determination of significance of a comparison of one or more biological samples. In the present illustration, reference samples taken from patients suffering from the disease state as well as reference samples taken from persons not exhibiting the disease state provide a basis for comparing statistically significant attributes of the patient derived sample. Example C provides an example of the output from the algorithm set forth in Example B. A user such as a medical profession is thus provided with a rapid means for screening a patient for the patient's propensity to develop a pathological condition, thus permitting early therapeutic intervention, or suggesting prophylactic treatment.

Other examples of the system and method according to the present invention are provided as examples below. These examples are not intended to be limiting, as other such embodiments are readily apparent to those skilled in the art in view of the teachings contained herein.

### EXAMPLE A

### Identification of Genetic Risk Factors

An exemplary sample population displaying evidence for the association between genetic variants and disease states is given at Table A. This study comprised 2400 individuals consisting of 800 dizygotic (DZ) sibling ( “sib”) pairs and 400 monozygotic (MZ) sib-pairs. The individuals were all female, ranged in age from approximately 20 to 70 years, and were all of Caucasian ethnicity. Age and zygosity were recorded for every sib-pair, and self-reported zygosity was confirmed by genotyping a standard marker set to confirm 50% or 100% allele sharing by DZ and MZ pairs, respectively.

TABLE A
Gene Markers for Disease States and their Phenotypes

| Gene | Phenotype | Clone ID No. |
|---|---|---|
| Human Gene SWISSPROT- | BMI, total fat mass, | 12252123 |
| ID:Q13608 PEROXISOME | waist size | |
| ASSEMBLY FACTOR-2 | | |
| (PAF-2) (PEROXISOMAL- | | |
| TYPE ATPASE 1) | | |
| (PEROXIN-6) - HOMO | | |
| SAPIENS (HUMAN), 980 | | |
| aa. | | |
| Novel | Serum concentration | 13373788 |
| | of lipoprotein A | |
| OLFACTORY-receptor- | Bone density and | 13019736 |
| like | gamma glutamyl | |
| | transpeptidase | |
| Human Gene Similar to | Serum bicarbonate | 12252120 |
| SWISSNEW-ID: P17709 | | |
| GLUCOKINASE (EC | | |
| 2.7.1.2) (GLUCOSE | | |
| KINASE) (GLK)- | | |
| SACCHAROMYCES | | |
| CEREVISIAE (BAKER'S | | |
| YEAST), 500 | | |
| aa. \|pcls:SWISSPROT- | | |
| ID:P17709 GLUCOKINASE | | |
| (EC 2.7.1.2) (GLUCOSE | | |
| KINASE) (GLK)- | | |
| SACCHAROMYCES | | |
| CEREVISIAE (BAKER'S | | |
| YEAST), 500 aa. | | |
| Galactosidase | Systolic blood | 12252108 |
| sialotransferase | pressure | |

The column labeled “GENE” indicates the reference gene under study, providing its name or identification if the sequence is publically available. The column labeled “PHENOTYPE” indicates the observed traits affected by gene expression products. The column labeled “CLONE ID NO.” indicates the proprietary Curagen designation for a clone containing the gene or a polymorphism thereof. These clones arc referenced in other applications.

Clinical measurements were made for 105 traits in categories including asthma and respiratory disease, biochemistry and endocrine function, bone density and osteoporosis, cardiovascular disease, diabetes, hypertension, obesity, immunology, rheumatology, oncology, CNS disorders, and dermatology. Each trait was measured for approximately 80% of the population.

Each trait was standardized to approximate a univariate standard normal distribution. For most traits, this involved calculating the trait mean and standard deviation, then subtracting the mean for each trait score and dividing by the standard deviation to yield a trait with zero mean and unit variance. For some traits, the distribution appeared log-normal, and a log transform was applied prior to the standardization. Genotypes were measured for each marker for at least 70% of the individuals with a discrepancy rate of 4% or less. Genotyping discrepancies do not increase the false-positive rate of a test, although they do increase the false-negative rate.

An individual was defined as informative if both the trait value and genotype were available. The total population was then partitioned into three groups: MZ pairs with both sibs informative, DZ pairs having both sibs informative, and unrelateds from both MZ pairs and DZ pairs in which only one sib was informative.

The terms nUnrel, nMZ, and nDZ refer to the number of unrelateds, number of MZ pairs, and number of DZ pairs, respectively; the total number of informative individuals is nUnrel+2 nMZ+2 nDZ.

The allele frequency of the minor allele (a number between 0 and 0.5) was determined as a weighted average in which unrelated individuals had a weight of 1, MZ individuals had a weight of 0.5, and DZ individuals had a weight of 0.75. These weightings account for genotypic correlation within a sib-pair.

The markers tested were all bi-allelic. The frequency of the minor allele, termed A, is denoted p, and the frequency of the major allele, termed allele B, is denoted q and equals 1−p.

A total of 6 tests of this nature were performed. The roughly 100 phenotypes tests correspond to approximately 20 independent tests because many of the phenotypes are correlated. This threshold corresponds to an approximate false-positive rate of 2% per marker tested.

Unrelated X₁, and p₁ are from the unrelated individuals and the MZ pairs. For the unrelateds, each individual yields a single sample of X₁ and p₁. For the MZ pairs, X₁ and p₁ were taken as the average of the two values. It would be preferable to account for the phenotypic correlation between MZ sibs as part of this test.

Mean Each DZ pair yields a single sample, with X₁ and p₁ equal to the mean phenotypic value and allele frequency of pair i.

Difference Each DZ pair yields a single sample, with X₁ and p₁ equal to the difference in phenotypic value and allele frequency between the first and second sib. This test is robust to stratification.

Non-parametric difference Each DZ pair yields a single sample, with p₁ equal to the difference in allele frequency between the first and second sib, and X₁ equal to 1, 0, or −1 if the phenotypic value of the first sib is greater than, equal to, or less than that of the second sib. This test is like a transmission disequilibrium test (TDT). Like the difference test, it is robust to stratification; it is also robust to non-normality and outliers, but is less sensitive to small effects than the difference test.

Total The total test combines the estimates of b from the unrelated, mean, and difference tests, which are statistically independent. A minimum variance estimator of b is built by weighting each of the three tests by the inverse of their sampling variance, and the variance of the combined estimator is the inverse of the sum of the inverse variances of the independent estimates. This test is more sensitive than either of the three independent tests in the absence of stratification, but is not as robust as the difference or non-parametric difference test in the presence of stratification.

Stratification The test statistic for the stratification test is the square of the difference of the estimates of b from the mean and difference tests, normalized by the sum of the variances of the two estimators, follows a χ² distribution with 1 degree of freedom. Large values of the test statistic indicate population stratification and that only the difference test and non-parametric difference test may be robust.

### EXAMPLE B

### Computer Program

The following computer readable program code, entitled “GOUDA” was written to execute an instruction set comprising the statistical analyses disclosed herein, that is designed to determine the genetic risk factor of a patient for the disease states or pathological conditions described in Example A, from input data obtained from biological samples. This code, written in the C language, can run on any computers having processors recognizing this language, for example but not limited to PowerPC™ based Apple® computers and Pentium™ or similar PC type computers.

In this example, data from reference and patient tissue samples are compared to determine the presence or absence of polymorphisms at a gene locus associated with or implicated in such disease states or disorders. The information is input into a computer system comprising a processing means for executing the following program or instruction set.

This program, provides a non-limiting example of one such type of computer readable instruction set for executing statistical and other data manipulations and calculations according to the disclosure provided. Other instruction sets can be writtin in similar computer readable formats, that perform essentially the same functions described, and are considered to be within the scope of this invention. For an example of language for developing computational algorithms according to the invention, see, W. H. Press, S. A. Teukolsky, W. T. Vetterling, and B. P. Flannery, Numerical Recipes in C, the Art of Scientific Computing, 2^{nd} edition, Cambridge University Press, New York, 1997, incorporated by reference.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

### EXAMPLE C

### Output of Program

The computer readable program provided in Example B, generates an output given below. This output is viewed on paper or electonic viewing means, e.g, a cathode ray terminal (CRT), light emitting diode (LED) display, or similar display means or projection means. The information output provided is thus a statistical analaysis of the information input into the program, and provides the user with information relating to genetic polymorphisms, and the presence of other genetic markers. The output information is thus correlated with disease states and pathological conditions, as deviation from control (healthy) samples or correlation with diseased samples, or similar comparisons to reference samples is detected.

[image]

[image]

[image]

[image]

[image]

[image]

[image]

[image]

[image]

[image]

[image]

### EQUIVALENTS

From the foregoing detailed description of the specific embodiments of the invention, it should be apparent that a unique procedure to evaluate genetic risk factors for a disease or pathology has been described. Although particular embodiments have been disclosed herein in detail, this has been done by way of example for purposes of illustration only, and is not intended to be limiting with respect to the scope of the appended claims that follows. In particular, it is contemplated by the inventor that substitutions, alterations, and modifications may be made to the invention without departing from the spirit and scope of the invention as defined by the claims.

We claim:

1. A system for detecting a genetic risk factor for a disease comprising:
a. an input module for entering data in computer readable format, said data comprising patient sample information and reference sample information;
b. a selection module instructing the system to select and read entered data;
c. an analyzing module instructing the system to perform biostatistical analyses of the entered data further comprising the patient sample information and reference sample information, thereby detecting statistically significant similarities or differences between the patient sample information and the reference sample information;
d. an association detection module instructing the system to correlate statistically significant similarities or differences between the patient sample information and the reference sample information with data relating to a pathological phenotype; and
e. a presenting module instructing the system to present to the user, the statistically significant similarities or differences between the patient sample information and the reference sample information, and the data relating to a pathological phenotype, wherein the user detects the patient's genetic risk factor for the disease.

2. The system of claim 1, wherein the gene sequence information is obtained by genotyping methods selected from the group consisting of oligonucleotide ligation, direct sequencing, mass spectroscopy, real time kinetic PCR, hybridization, pyrosequencing, fragment polymorphisms, and fluorescence depolarization.

3. The system of claim 1, wherein the patient derived biological sample is obtained from tissues selected from the group consisting of nucleated cells, blood, hair folicles, buccal scrapings, saliva, organ biopsies, and semen.

4. The system of claim 1, wherein the polymorphism is predictive of a risk for a sibling of the patient to develop the genetic disease.

5. The system of claim 1, wherein the polymorphism is predictive of a risk for an offspring of the patient to develop the genetic disease.

6. A method for detecting a genetic risk factor for a disease comprising:
a. obtaining a patient derived biological sample, wherein the patient derived sample contains a detectable marker correlated with a disease state or pathological condition;
b. obtaining from said biological sample data comprising patient sample information further comprising a polymorphism in a marker, relative to the sequence of a wild-type marker; and and wherein the polymorphism is correlated with a disease state or pathological condition; and
c. detecting an association between the patient sample information and the disease state, thereby detecting the genetic risk factor for the patient to develop the disease, wherein detecting said association further comprises performing biostatistical analysis on the genetic locus.

7. The method of claim 6, wherein the patient sample information is obtained by genotyping methods selected from the group consisting of oligonucleotide ligation, direct sequencing, mass spectroscopy, real time kinetic PCR, hybridization, pyrosequencing, fragment polymorphisms, and fluorescence depolarization.

8. The method of claim 6, wherein the patient sample information is obtained from tissues selected from the group consisting of nucleated cells, blood, hair folicles, buccal scrapings, saliva, organ biopsies, and semen.

9. The method of claim 6, wherein the polymorphism is predictive of a risk for an offspring of the patient to develop the genetic disease.

10. The method of claim 6, wherein the polymorphism is predictive of a risk for a sibling of the patient to develop the genetic disease.

11. A method for detecting a genetic risk factor for a disease comprising:
a. obtaining a patient derived biological sample, wherein the patient derived sample contains a detectable marker correlated with a disease state or pathological condition;
b. obtaining from said biological sample data comprising patient sample information further comprising a polymorphism in a marker, relative to the sequence of a wild-type marker; and and wherein the polymorphism is correlated with a disease state or pathological condition; and
c. detecting an association between the patient sample information and the disease state, thereby detecting the genetic risk factor for the patient to develop the disease, wherein detecting said association further comprises performing Hardy-Weinberg tests and association tests on the genetic locus.

12. The method of claim 11, wherein the patient sample information is obtained by genotyping methods selected from the group consisting of: oligonucleotide ligation, direct sequencing, mass spectroscopy, real time kinetic PCR, hybridization, pyrosequencing, fragment polymorphisms, and fluorescence depolarization.

13. The method of claim 11, wherein the patient sample information is obtained from tissues selected from the group consisting of nucleated cells, blood, hair folicles, buccal scrapings, saliva, organ biopsies, and semen.

14. The method of claim 11, wherein the polymorphism is predictive of a risk for an offspring of the patient to develop the genetic disease.

15. The method of claim 11, wherein the polymorphism is predictive of a risk for a sibling of the patient to develop the genetic disease.

16. A processor readable medium, said processor readable medium comprising:
a. a first processor readable program code for causing a processor to select select and read entered data, said data further comprising patient sample information and reference sample information;
b. a second processor readable program code for causing a processor to perform biostatistical analyses of the entered data further comprising the patient sample information and reference sample information, thereby detecting statistically significant similarities or differences between the patient sample information and the reference sample information;
c. a third processor readable program code for causing a processor to correlate statistically significant similarities or differences between the patient sample information and the reference sample information with data relating to a pathological phenotype; and
d. a fourth processor readable program code for causing a processor to present to the user, the statistically significant similarities or differences between the patient sample information and the reference sample information, and the data relating to a pathological phenotype, thus permitting the user to detect the patient's genetic risk factor for the disease.

17. The processor readable medium of claim 16, wherein the first processor readable program code allows a user to input patient sample information obtained by genotyping methods selected from the group consisting of oligonucleotide ligation, direct sequencing, mass spectroscopy, real time kinetic PCR, hybridization, pyrosequencing, fragment polymorphisms, and fluorescence depolarization.

18. The processor readable medium of claim 16, wherein the first processor readable program code allows a user to input patient sample information obtained from tissues selected from the group consisting of nucleated cells, blood, hair folicles, buccal scrapings, saliva, organ biopsies, and semen.

19. The processor readable medium of claim 16, wherein the second processor readable program code for causing a processor to perform biostatistical analyses detects a polymorphism in a patient gene sequence that is is predictive of a risk for an offspring of the patient to develop the genetic disease.

20. The processor readable medium of claim 16, wherein the second processor readable program code for causing a processor to perform biostatistical analyses detects a polymorphism in a patient gene sequence that is is predictive of a risk for a sibling of the patient to develop the genetic disease.
